(19) United States
(12) Patent Application Publication
(10) Pub. No.: US 2005/0187599 A1
(43) Pub. Date: Aug. 25, 2005

(54) Method and apparatus for controlled contraction of soft tissue

(76) Inventors: Hugh Sharkey, Redwood City, CA (US); Gary Fanton, Portola Valley, CA (US)

Correspondence Address: MORRISON & FOERSTER, LLP, 555 WEST FIFTH STREET, SUITE 3500, LOS ANGELES, CA, 90013-1024, US

(21) Appl. No.: 11/112,164

(22) Filed: Apr. 21, 2005

Related U.S. Application Data
(63) Continuation of application No. 08/714,987, filed on Sep. 17, 1996.
Application No. 08/714,987 is a continuation-in-part of application No. 08/637,095, filed on Apr. 24, 1996, now Pat. No. 6,482,204.
Application No. 08/637,095 is a continuation of application No. 08/389,924, filed on Feb. 16, 1995, now Pat. No. 5,569,242.
Application No. 08/389,924 is a continuation of application No. 08/238,862, filed on May 6, 1994, now Pat. No. 5,458,596.

(51) Int. Cl. A61F 7/00; A61F 7/12

(52) U.S. Cl. 607/98; 607/99

(57) ABSTRACT

An apparatus for effecting change in at least a portion of a selected site of a collagen containing tissue that is at least partially adjacent to a fluid medium. The apparatus includes an energy delivery device configured to deliver a level of energy to the selected site of the collagen containing tissue. The energy delivery device includes a distal portion where a sensor is positioned. The sensor provides a signal indicative of the thermal energy content of at least the selected site of the collagen containing tissue and the adjacent fluid medium to a feedback control unit. The signal is received by the feedback control system which adjusts the level of energy supplied to the energy delivery.

### RELATED INVENTIONS

This application is a continuation of Ser. No. 08/714,987, filed Sep. 17, 1996, entitled METHOD AND APPARATUS FOR CONTROLLED CONTRACTION OF SOFT TISSUE, which is a continuation in part of Ser. No. 08/637,095, filed Apr. 24, 1996, now U.S. Pat. No. 6,482,204, entitled METHOD AND APPARATUS FOR CONTROLLED CONTRACTION OF SOFT TISSUE, which is a continuation of Ser. No. 08/389,924, filed Feb. 16, 1995, now U.S. Pat. No. 5,569,242, entitled METHOD AND APPARATUS FOR CONTROLLED CONTRACTION OF SOFT TISSUE, which is a continuation of Ser. No. 08/238,862, filed May 6, 1994, now U.S. Pat. No. 5,458,596, entitled METHOD AND APPARATUS FOR CONTROLLED CONTRACTION OF SOFT TISSUE, each of which is hereby incorporated herein by reference in its entirety.

### CROSS-REFERENCE TO RELATED APPLICATIONS/BACKGROUND OF THE INVENTION

1. Field of the Invention

This invention relates generally to a method and apparatus for delivering thermal energy to a selected collagen containing tissue and effecting a contraction of at least a portion of the collagen containing tissue, and more particularly to a method and apparatus for contracting a collagen containing tissue that is at least partially adjacent to a fluid medium.

2. Description of the Related Art

Instability of peripheral joints has long been recognized as a significant cause of disability and functional limitation in patients who are active in their daily activities, work or sports. Diarthrodial joints of the musculoskeletal system have varying degrees of intrinsic stability based on joint geometry and ligament and soft tissue investment. Diarthrodial joints are comprised of the articulation of the ends of bones and their covering of hyaline cartilage surrounded by a soft tissue joint capsule that maintains the constant contact of the cartilage surfaces. This joint capsule also maintains within the joint the synovial fluid that provides nutrition and lubrication of the joint surfaces. Ligaments are soft tissue condensations in or around the joint capsule that reinforce and hold the joint together while also controlling and restricting various movements of the joints. The ligaments, joint capsule, and connective tissue are largely comprised of collagen.

When a joint becomes unstable, its soft tissue or bony structures allow for excessive motion of the joint surfaces relative to each other and in directions not normally permitted by the ligaments or capsule. The two main forms of joint instability are called subluxations and dislocations. A subluxation occurs when one surface of a joint slides out of position relative to the other surface while retaining some contact between the surfaces. A dislocation occurs when one surface of the joint completely disengages and loses contact with the opposing surface. Generally, joints with a larger range of motion have more inherently loose soft tissue investments surrounding the joint and as a result are more prone to instability than others. For example, the shoulder (glenohumeral) joint has the greatest range of motion of all peripheral joints and has long been recognized as having the highest subluxation and dislocation rate.

Instability of the shoulder can not only occur congenitally and developmentally but also traumatically. Furthermore, this instability often becomes recurrent and requires surgical repair. In fact, subluxations and dislocations are a common occurrence and cause for a large number of orthopedic procedures each year. Joints which require repair are characterized by symptoms which include pain, instability, weakness and limitation of function. If the instability is severe and recurrent, functional incapacity and arthritis may result. Surgical attempts are directed toward tightening soft tissue restraints which have become loose. These procedures are typically performed through open surgical approaches that often require hospitalization and prolonged rehabilitation programs.

More recently, endoscopic (arthroscopic) techniques for achieving these same goals have been explored with variable success. Endoscopic techniques have the advantage of being performed through smaller incisions and are usually less painful, performed on an outpatient basis, are associated with less blood loss and lower risk of infection and have a more cosmetically acceptable scar. Recovery is often faster postoperatively than using open techniques. However, it is often more technically demanding to advance and tighten capsule or ligamentous tissue arthroscopically because of the difficult access to pathologically loose tissue and because it is very hard to determine how much tightening or advancement of the lax tissue is clinically necessary. In addition, fixation of advanced or tightened soft tissue is more difficult arthroscopically than through open surgical methods.

Collagen containing tissue is ubiquitous in the human body and provides the cohesiveness of the musculoskeletal system, the structural integrity of the viscera as well as the elasticity of integument. Collagen also demonstrates unique characteristics not found in other tissues. A previously recognized property of collagen is shrinkage of collagen fibers when elevated in temperature. Collagen fibrils are at their greatest length in the native state of a triple helix. Thermal energy to the collagen molecules disrupts the bonds which stabilize the triple helix. The loss of the triple helix structure causes the fibrils to decrease in length or contract, giving the collagen containing tissue the appearance of contracting. The degree of contraction is a function of both the height of temperature elevation as well as the length of temperature elevation. Thus, the same degree of contraction may be achieved by a high temperature elevation of short duration or by a lower temperature elevation for an extended duration.

Investigators have taken advantage of the unique collagen features to effect positive changes in non-vascularized collagen containing structures. For instance, the use of infrared laser energy to shrink collagen in the cornea of the eye relates to laser keratoplasty and has been described by Sand in U.S. Pat. No. 4,976,709. Further, radio frequency (RF) electrical current has been used to reshape the cornea. Such shaping has been reported by Doss in U.S. Pat. Nos. 4,325,529 and 4,381,007.

The capsule of the shoulder joint consists of a synovial lining and three well defined layers of collagen. The fibers of the inner and outer layers extend in a coronal access from the glenoid to the humerus. The middle layer of the collagen extends in a sagittal direction, crossing the fibers of the other two layers. The relative thickness and degree of intermingling of collagen fibers of the three layers vary with different portions of the capsule. The ligamentous components of the capsule are represented by abrupt thickenings of the inner layer with a significant increase in well organized coarse collagen bundles in the coronal plane. The capsule functions as a hammock-like sling to support the humeral head. In pathologic states of recurrent traumatic or developmental instability this capsule or pouch becomes attenuated and the capsule capacity increases secondary to capsule redundance. In cases of congenital or developmental multi-directional laxity, the ratio of type III to type I collagen fibers is often larger than usual. An apparatus capable of shrinking the collagen containing tissue in the shoulder may eliminate many of these instabilities. Further, if this apparatus could be used endoscopically, many of the problems with current endoscopic techniques would be eliminated since fixation, tightening and advancement would no longer be required.

The use of endoscopic devices which simply heat the collagen containing tissue are not satisfactory because of the delivery of uncontrolled energy. High temperatures can cause cell necrosis and may damage the tissue.

There is a need for a method and apparatus which causes collagen containing tissues to contract while minimizing cell necrosis and damage to the tissue as well as other organs or bodies which may be present, more particularly, for joints and shoulder capsules. There is a need for a method and apparatus capable of causing a collagen containing tissue site at least partially adjacent to a fluid media to contract a selected amount without damaging the tissue of the site or any of the surrounding tissues or bodies whether they contain collagen or not.

### SUMMARY OF THE INVENTION

It is an object of the present invention to provide a method and apparatus configured to contract at least a portion of a selected site of a collagen containing tissue.

Another object of the present invention is to provide a method and apparatus configured to deliver sufficient energy to a selected site of a collagen containing tissue to produce a contraction of at least a portion of the selected site.

Still another object of the present invention is to provide a method and apparatus configured to deliver sufficient energy to a selected site of a collagen containing tissue to effect an increase in the thermal energy content of the selected site.

Yet another object of the present invention is to provide a method and apparatus configured to deliver sufficient energy to a selected site of a collagen containing tissue to effect an increase in the temperature of the selected site to a pre-determined level.

A further object of the present invention is to provide a method and apparatus configured to deliver sufficient energy to a selected site of a collagen containing tissue such that the temperature of the selected site increases to a pre-determined level and remains at or near that level for a selected period of time.

Yet a further object of the present invention is to provide a method and apparatus configured to deliver sufficient energy to a selected site of a collagen containing tissue to create a contraction of collagen fibers.

Still another object of the present invention is to provide a method and apparatus with a feedback control device configured to deliver sufficient and controllable energy to a selected site of a collagen containing tissue.

Another object of the present invention is to provide a method and apparatus with a feedback control device configured to deliver sufficient energy to a selected site of a collagen containing tissue positioned at least partially adjacent to a fluid medium to contract at least a portion of the selected site and produce a thermal feedback signal representative of a composite of the thermal energy contents of at least a portion of the selected site and at least a portion of the adjacent fluid medium.

Another object of the present invention is to provide a method and apparatus with a feedback control device configured to deliver sufficient thermal energy to a selected site of a collagen containing tissue of an unstable joint at least partially positioned adjacent to a fluid medium and at least partially repair the instability of the joint.

These and other objects of the invention are obtained with an apparatus for effecting change in at least a portion of a selected site of a collagen containing tissue that is at least partially adjacent to a fluid medium. The apparatus includes an energy delivery device configured to deliver a level of energy to the selected site of the collagen containing tissue. The energy delivery device includes a distal portion where a sensor is positioned. The sensor provides a signal indicative of the thermal energy content of at least the selected site of the collagen containing tissue and the adjacent fluid medium to a feedback control unit. The signal is received by the feedback control system which adjusts the level of energy supplied to the energy delivery device and delivered to the selected site based on the signal received from the sensor.

In another embodiment, the apparatus includes an energy delivery device configured to produce a selected thermal distribution in the selected site of the collagen containing tissue to effect a controllable contraction of at least a portion of the collagen fibers. The energy delivery device includes a sensor positioned at a distal portion of the energy delivery device. A feedback control device is coupled to the sensor. A position of the sensor, a geometry of the distal portion of the energy delivery device and the feedback control system provide a controllable energy delivery to the selected site of the collagen containing tissue.

The energy delivery device is configured to deliver energy from the distal portion to the selected site of the collagen containing tissue. The selected site absorbs at least a portion of the delivered energy and the thermal energy content and temperature of the selected site are increased. As the thermal energy content of the selected site is increased, thermal energy is conducted to the collagen fibers of the selected site. Collagen fibers exposed to sufficient thermal energy at least partially lose their triple helix shape and contract. Thus, the delivery of energy to the selected site causes the temperature and the thermal energy content of the selected site to increase and create a contraction of at least a portion of the collagen containing tissue site.

In one embodiment, the sensor is located within the distal portion. During surgery, the distal portion is preferably placed in contact with a portion of the selected site and the fluid medium adjacent to the selected site. Because of this contact, the thermal energy from the selected site and the adjacent fluid medium will conduct through the thermally conductive sections of the distal portion to the sensor. The magnitude of the resulting signal represents a composite of the thermal energy contents of the selected site and the adjacent fluid medium.

The sensor provides a signal which is representative of the thermal energy contents of a portion of the fluid medium adjacent to the selected site as well as at least a portion of the selected site. As a surgeon moves the distal portion about a selected area, it is possible for a surgeon to bring the distal portion into contact with a selected site which has previously been elevated to the desired temperature for the desired period of time. This second application of energy may quickly elevate the temperature enough to cause cell necrosis or cause the temperature at the selected site to remain elevated for longer than the desired period required for the desired level of collagen contraction.

Since the magnitude of the signal provided by the sensor partially represents the thermal energy of the fluid medium, the apparatus is responsive to changes in the thermal energy content of the fluid medium. Due to the nature of delivering energy to a selected site, thermal energy is more disperse in the fluid medium. Because the apparatus responds to thermal energy in the fluid medium, the apparatus reduces cell necrosis resulting from successive applications of energy to the selected site. Stray contractions are contractions which occur away from the selected site due to the fluid medium becoming elevated in temperature for an extended period of time. Further, response of the apparatus to thermal energy in the fluid medium can also reduce stray contractions.

### DESCRIPTION OF THE DRAWINGS

FIG. 1 is a perspective plan view of an embodiment of the present invention illustrating an apparatus for contracting collagen containing tissue.

FIG. 2 is a perspective plan view of an embodiment of the present invention illustrating an apparatus coupled to an energy source for contracting collagen containing tissue.

FIG. 3 is a perspective plan view of an embodiment of the present invention illustrating an apparatus for contracting collagen containing tissue a desired amount in contact with a collagen containing tissue.

FIG. 4 is a perspective plan view of an embodiment of the present invention illustrating an apparatus for contracting collagen containing tissue a desired amount delivering heat to a selected site within a selected area.

FIG. 5 illustrates the positioning of a distal end of an energy delivery device while delivering energy to a selected tissue site and a portion of an adjacent fluid medium and the measurement of a composite temperature.

FIG. 6 is a cross-sectional view of a distal portion of the energy delivery device with a sensor positioned in interior of the distal portion.

FIG. 7 is a perspective plan view of an embodiment of the present invention illustrating an apparatus for contracting collagen containing tissue a desired amount where the energy delivery surface is a composite construction.

FIG. 8 is a schematic of an embodiment of the present invention illustrating a feedback control system.

FIGS. 9(a)-(d) are perspective plan views of different embodiments of the present invention illustrating several apparatus, each configured to provide a signal from a sensor such that the signal represents thermal energies of different surfaces or mediums.

FIG. 10 is a perspective plan view of an embodiment of the present invention illustrating an apparatus with a handpiece, energy delivery device and an operating cannula according to the present invention.

FIG. 11 is a perspective plan view of an embodiment of the present illustrating an invention apparatus including an insulating layer for preventing damage to surrounding tissues, organs or bodies.

FIG. 12 is a perspective plan view of an embodiment of the present invention illustrating an apparatus including a handpiece, an energy delivery device and a sleeve that slides across the surface of the energy delivery device to vary the amount of energy delivery device conductive surface.

FIG. 13 is a perspective plan view of an embodiment of the present invention illustrating an apparatus including a thermal insulating layer which can he positioned to specify the surface of the distal end section from which the sensor is able to detect thermal energy.

FIG. 14 is a sectional view of an embodiment of the present invention illustrating a deflected energy delivery device with a resistive heating element positioned in an interior lumen of the energy delivery device.

FIG. 15 is a perspective plan view of an embodiment of the present invention illustrating an energy delivery device with a steering wire positioned on the exterior of the energy delivery device.

FIG. 16 is a sectional view of an embodiment of the present invention illustrating an energy delivery device with a lumen and a plug that is attached to the energy delivery device distal end.

FIG. 17 is a sectional view of an embodiment of the present invention illustrating an energy delivery device with an oval cross section and a heating zone in the tissue.

FIG. 18 is a sectional view of an embodiment of the present invention illustrating a handle, energy delivery device, operating cannula and a viewing scope, with the viewing scope and energy delivery device positioned in the operating cannula.

FIG. 19 is a cross sectional view of an embodiment of the present invention illustrating a device of FIG. 18, taken along the lines 19-19.

### DETAILED DESCRIPTION OF THE PREFERRED EMBODIMENTS

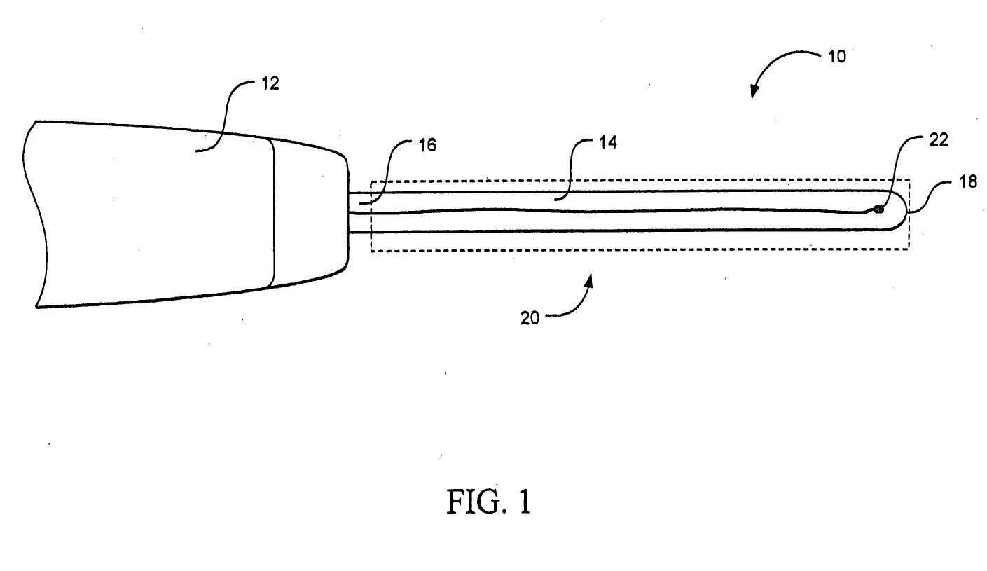

Referring now to FIG. 1, an apparatus for contracting collagen containing tissue to a desired level is generally denoted as 10. Apparatus 10 includes a handpiece 12 that is preferably made of a thermal insulating material, or an electrode that is electrically insulated. Types of such insulating materials are well known to those skilled in the art. An energy delivery device 14 is coupled to handle 12 at a proximal end 16 of energy delivery device 14, and may be attached thereto. A distal end 18 of energy delivery device 14 includes a distal portion 20 which may have a geometry that delivers a controlled amount of energy to tissues in order to achieve a desired level of contraction of collagen fibers in a collagen containing tissue. Located at distal portion 20 is one or more sensors 22 which provide a signal whose magnitude is representative of the amount of thermal energy sensed.

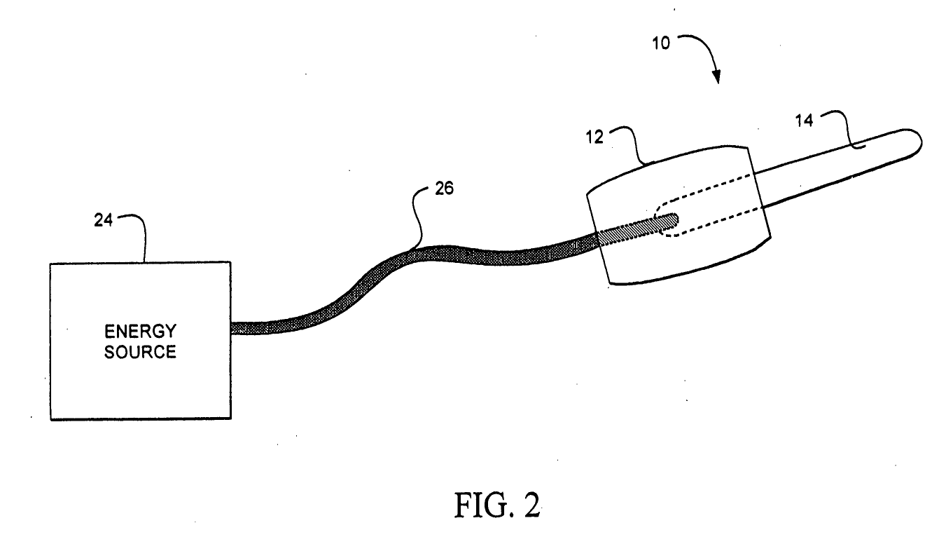

As shown in FIG. 2, energy is supplied from an energy source 24 through a cable 26 to energy delivery device 14. Since several types of energy can cause an elevation in the temperature of a collagen containing tissues 28. Energy source 24 can include but is not limited to RF, microwave, ultrasonic, coherent and incoherent light, thermal transfer, and resistance heating.

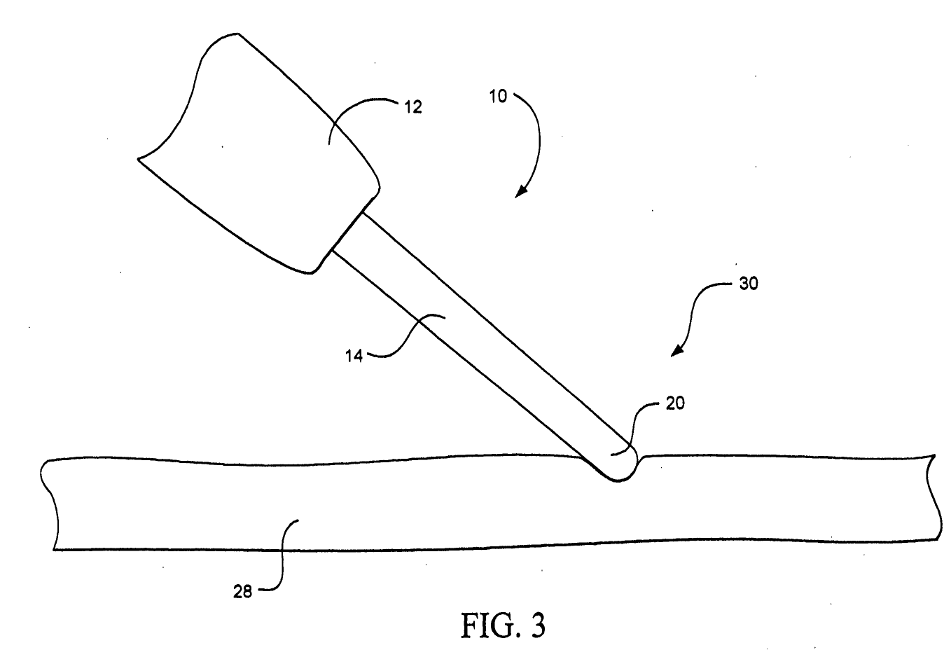

As illustrated in FIG. 3, distal portion 20 is configured to be positioned adjacent to a collagen containing tissue 28 which is at least partially adjacent to a fluid medium 30. Appropriate collagen containing tissues 28 can include but are not limited to vascularized densely collagenous structures such as tendons, ligaments, joints capsules and the like. Distal portion 20 is preferably in contact with collagen containing tissue 28. Fluid medium (gas, liquid, or a combination) 30 may be flowing as would result from irrigating collagen containing tissue 28 or it may be substantially less dynamic or non-moving. Further, fluid medium 30 need only be partially fluid and contain bone, portions of organs or other bodies and the like.

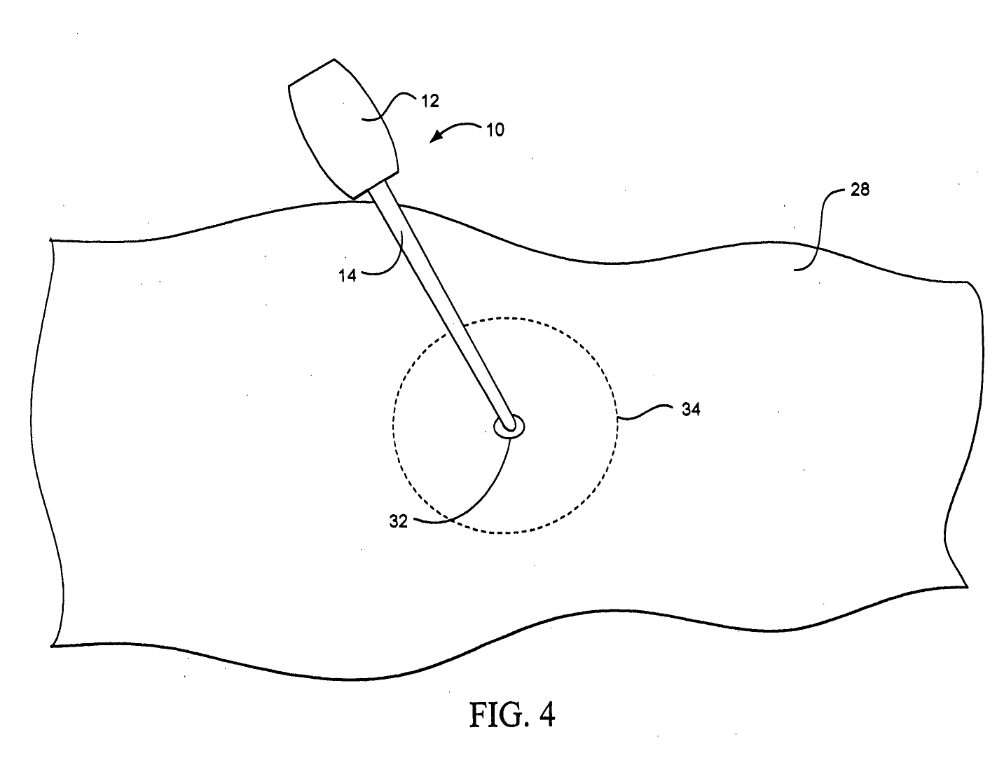

Referring now to FIG. 4, energy delivery device 14 is configured to deliver energy from distal portion 20 to a selected site 32 of the collagen containing tissue 28. Selected site 32 receives at least a portion of the delivered energy. Once the energy is delivered it becomes thermal energy causing the thermal energy content and the temperature of selected site 32 to increase. As the thermal energy content of selected site 32 is increased, thermal energy is conducted to the collagen fibers in and around selected site 32. Collagen fibers exposed to sufficient thermal energy loose their triple helix shape. Since the triple helix shape of collagen fibers is the longest shape for collagen fibers, fibers which loose their triple helix shape will contract. Thus, the delivery of energy to selected site 32 causes the temperature and the thermal energy content of selected site 32 to increase and effects collagen fibre contractions. The collagen fiber contraction results in a contraction of collagen containing tissue 28.

Energy delivery device 14 is configured to deliver a level of energy to selected site 32. Sensor 22 provides a signal indicative of a composite temperature of at least selected site 32 and at least a portion of at least a portion of adjacent fluid medium 30 to a feedback control unit. The signal is received by a feedback control system which adjusts the level of energy supplied to energy delivery device 14 and delivered to selected site 32 based on the signal received from sensor 22.

Throughout the treatment, it is often be desirable to effect contractions in a selected area 34 which is larger than a selected site 32. Further, may be desirable to elevate the temperature of the selected site 32 or selected area 34 to a desired average temperature for a specified period of time. There are several methods available for achieving these results. For instance, one embodiment is to “paint” distal portion 20 across selected area 34 by continually moving distal portion 20 over the surface of the selected area 34 so that the entire selected area 34 is covered. Selected area 34 can then be brought to the desired temperature and retained at that temperature by continually moving distal portion 20 over selected area 34. In another embodiment, distal portion 20 is left at selected site 32 until the desired temperature is obtained for the desired time. Distal portion 20 is then moved to another selected site 32 for a desired time. This pattern is repeated until the entire selected area 34 is covered. A combination of these techniques may also be used.

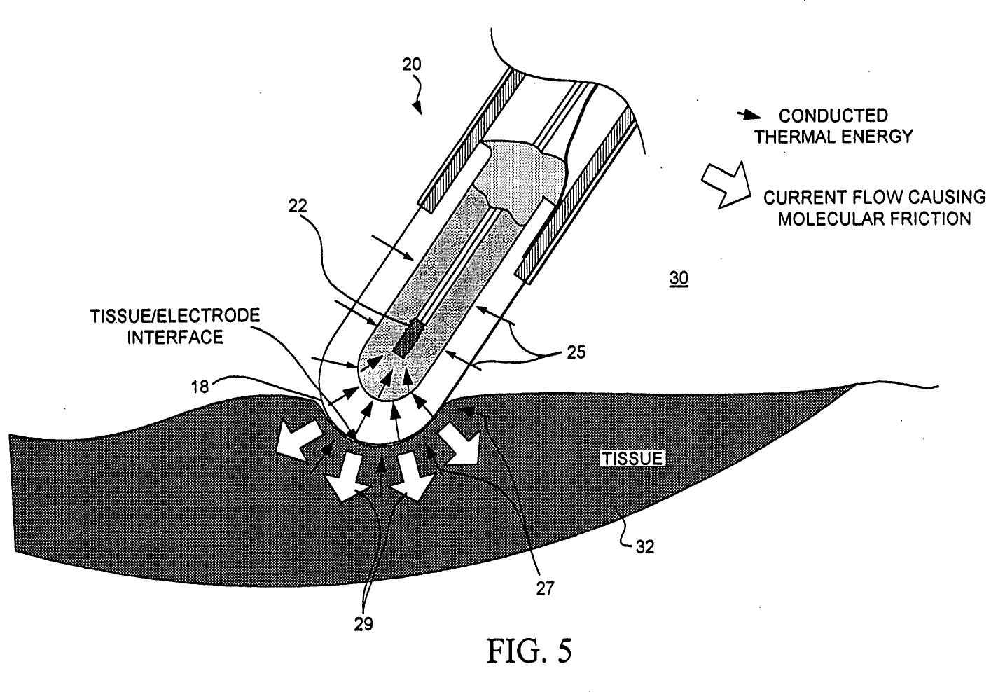

Referring now to FIG. 5, the composite temperature is a combination of at least two different temperatures in some ratio. One temperature 25 is from at least a portion of adjacent fluid medium 30 and another temperature 27 of at least a portion of selected tissue site 32. This ratio is a function of different parameters including but not limited to the size, shape, dimensions and geometry of a thermal energy delivery surface of energy delivery device 14, the portion of the thermal energy delivery surface that is in contact with adjacent fluid medium 30 and selected tissue site 32, the location of sensor 22 in relationship to the thermal energy delivery surface. Current flow 29 which creates molecular friction, and conducted thermal energy are greater in selected tissue site 32 than in adjacent fluid medium 30 due to the higher resistance of the tissue.

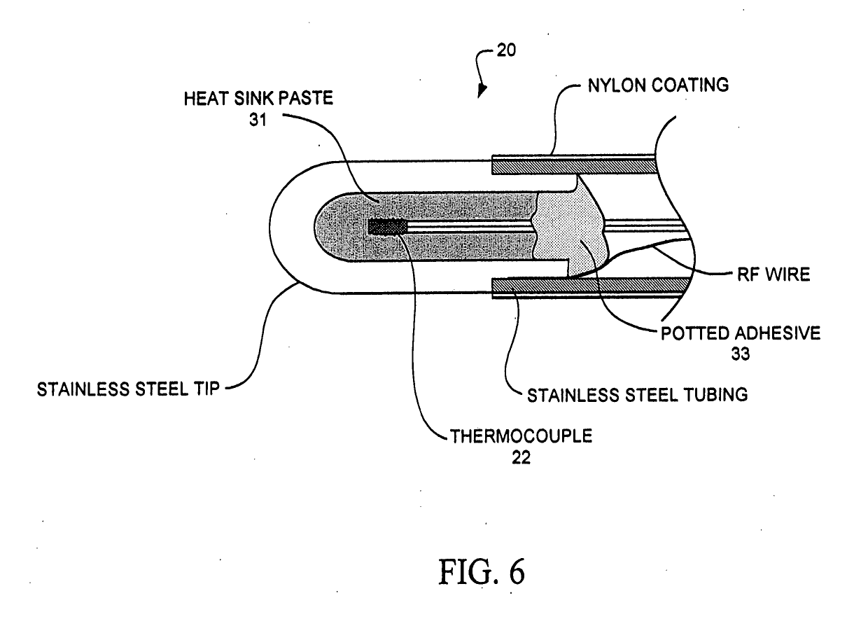

One embodiment of distal portion 20 is illustrated in FIG. 6. Distal portion 20 of energy delivery 14 includes sensor 22 positioned in an interior of distal portion 20. A thermally conductive material 31 at least partially surrounds sensor 22 and a potting compound 33 is included. Distal end 18 is made of stainless steel, and a nylon coating is positioned at an exterior surface of distal portion 20.

At the thermal energy delivery device fluid medium interface there is less resistance and a hydro dynamic force which contribute to a lower reflected temperature. At the tissue interface there is a static conductive situation with a higher resistance producing higher reflective temperature at the interface.

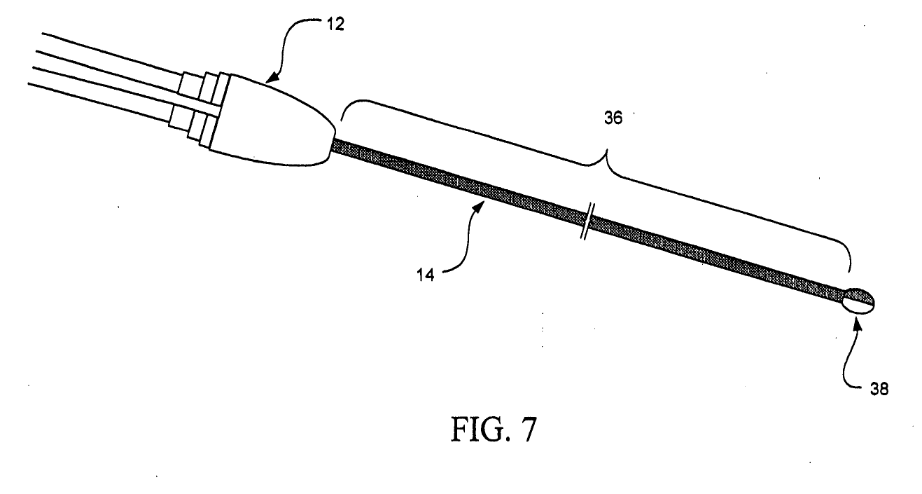

Energy delivery device 14 can be made of a number of different materials including but not limited to stainless steel, platinum, other noble metals and the like. Energy delivery device 14 can be made of a memory metal, such as nickel titanium, commercially available from Raychem Corporation, Menlo Park, Calif. Energy delivery device 14 can also be a composite construction whereby different sections are constructed from different materials. Further, it may be desirable for delivery device 14 to be a composite of a first material 36 which is not conductive to the type of energy being delivered and a second material 38 which is conductive to the type of energy being delivered as shown in FIG. 7. Such a construction permits treatment in locations where there are tissues, organs or other bodies present which the surgeon does not wish to expose to the delivered energy. For example, when energy delivery device 14 is introduced into a joint where it is desirable to treat a specific section of the joint and avoid delivery of energy outside of that section, an energy delivery device 14 partially constructed of non-conducting material 36 can permit treatment.

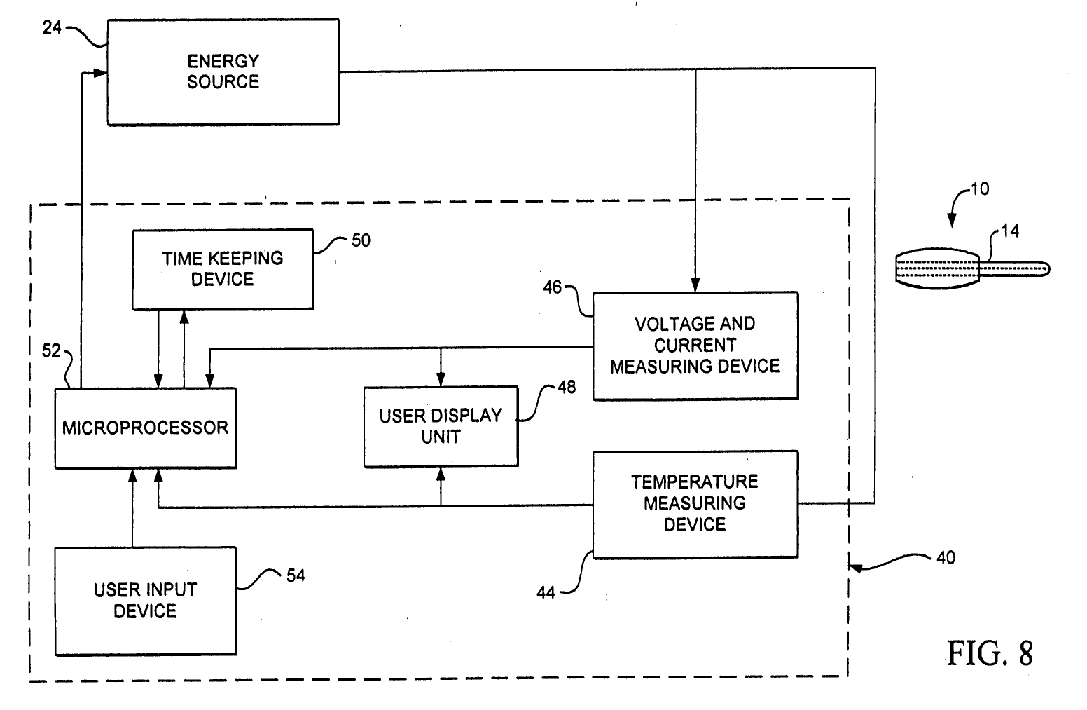

One embodiment of an open or closed loop feedback control system 40 is shown in FIG. 8. The physician can, if desired, override the closed or open loop feedback control system 40. The feedback control system 40 includes an energy source 24, (including but not limited to a RF source), a temperature measuring device 44, a voltage and current measuring device 46, a user display unit 48, a timekeeping device 50, a microprocessor 52 and a user input device 54.

Energy source 24 supplies energy to energy delivery device 14 for delivery to selected site 32. The voltage and current supplied to the energy delivery device 14 are measured on voltage and current measuring device 46 and can display these to the user on user display unit 48. Temperature measuring device 44 measures the temperature at sensor 22, including the temperature of adjacent fluid medium 30 and selected tissue site 32. The temperature measured by temperature measuring device 44 can be displayed to the user on the user display unit 48.

In one embodiment a signal produced by the sensor 22 is received by a feedback control system 40. Feedback control system 40 monitors the signal produced by sensor 22 and adjusts the amount of energy or current supplied to energy delivery device 14 according to the magnitude of the signal. Energy is supplied to apparatus 10 at a particular rate. The rate of energy delivery can be expressed as power. Power supplied to energy delivery device 14 is adjusted so the temperature at sensor 22 is elevated to a temperature which is desired by the user and is input to the feedback control system 40. Once the desired temperature is reached, power is adjusted so that the temperature at sensor 22 has minor fluctuations but averages to the desired temperature over time. Thus, the feedback control system 40 maintains the desired temperature at the sensor 22 and correspondingly at the selected site 32.

In another embodiment, feedback control system 40 also monitors time. In this embodiment both time and temperature are inputs. Thus, once the temperature at sensor 22 is elevated to the desired temperature, feedback control system 40 tracks the length of time sensor 22 averages the desired temperature. Once the temperature at sensor 22 averages the desired temperature for the desired time, feedback control system 40 may either stop the delivery of energy or it may inform the user on a user display screen (not shown). Thus, feedback control system 40 can be used to maintain the desired temperature at selected site 32 for the desired time.

In one embodiment, microprocessor 52 monitors voltage, current and temperature. Microprocessor 52 can calculate the power supplied to energy delivery device 14 from the current and voltage and can display the power on the user display unit 48. Microprocessor 52 can also monitor and control a timekeeping device 50. The microprocessor 52 can signal timekeeping device 50 to begin or stop tracking time. While timekeeping device 50 is tracking time and microprocessor 52 can monitor the passage of time. Microprocessor 52 also receives input from a user input device 54. User input device 54 allows a user to program microprocessor 52 or input information such as the desired temperature or the desired time.

Energy source 24 includes circuitry for modulating the power supplied to the energy delivery device 14 according to a signal received from microprocessor 52, thus, microprocessor 52 can control the power supplied to the energy delivery device 14. Microprocessor 52 is programmed to adjust the power supplied to energy delivery device 14 so that the temperature at sensor 22 is maintained at the desired temperature for the desired time. The program takes into account at least the desired time, temperature and desired temperature in making these adjustments.

Feedback control system 40 is used to obtain the desired degree of contraction by maintaining selected site 32, at a desired temperature for a desired time. It has been shown that temperatures of 45 to 90 degrees C. can cause collagen fibers contractions. It has also been shown that the degree of collagen fiber contraction is controlled by how long the temperature is elevated as well as how high it is elevated. Thus, the same degree of contraction can be obtained by exposing selected site 32 to a high temperature for a short period of time or by exposing selected site 32 to a lower temperature for a longer period of time. A preferred range for desired temperatures is about 45 to 75 degrees C., still a more preferred range is 45 to 65 degrees C. Before treatment, the surgeon evaluates the characteristics of the selected site 32 to determine what degree of contraction is necessary and also whether it is appropriate to treat the selected site 32 with a high temperature for a low period of time or lower temperature for a long period of time. The surgeon then enters into the user input device 54 the desired temperature and the desire time. Feedback control system 40 uses this information to control the delivery of energy to selected site 32 which results in a controlled contraction of collagen containing tissue fibers. The controlled collagen fiber contraction allows for a desired degree of collagen containing tissue contraction.

Additionally, feedback control system 40 can be used to effect how deeply within the collagen containing tissue 28 the collagen fiber contractions occur. For instance, elevating the temperature of selected site 32 to the low end of the range effects contractions near the surface of collagen containing tissue 28. Elevating the temperature to the high end of the range effects contractions deeper within collagen containing tissue 28. Thus, if the surgeon is dealing with a very thin collagen containing tissue 28 which is adjacent to tissue which may be damaged by elevated temperatures, the surgeon may choose to elevate the temperature of selected area 34 to a low temperature for longer periods of time. However, if collagen containing tissue 28 is thicker, the surgeon may choose higher temperatures to effect contractions deeper in collagen containing tissue 28. Thus, the choice of the desired temperature can control the thermal energy distribution and thus the depth of contractions.

Feedback control system 40 further allows apparatus 10 to minimize and even prevent cell necrosis (ablation) resulting from exposure to high temperatures. High temperatures can cause excessive destruction and disintegration of the collagen fibrillar patterns and cell necrosis. Since feedback control system 40 can maintain the temperature of sensor 22 at a desired temperature, the temperature at selected site 32 does not exceed ablation temperature.

Further, feedback control system 40 can prevent overshoots which may cause cell necrosis. Overshoots occur while raising the temperature of selected site 32 or selected area 34 to the desired level and temporarily surpassing that level. Some overshoot of the desired temperature will be inherent in most embodiments of feedback control systems, however, it is possible to cause cell necrosis or dissociation if the overshoot is high enough or of long enough duration. Feedback control system 40 reduces overshoots by reducing the rate of energy delivery once the selected site 32 temperature is near the desired level. Thus, when energy is first delivered to a selected site 32, there can be a high rate of energy delivery, however, once the temperature of the selected site is nearing the desired range, the rate of energy delivery is reduced in order to prevent an overshoot. Programming this ramping down effect into a feedback control system 40 is well known to those in the art of feedback control. Note: Some overshoots are ok, but the average temp must fall within a non-dilative range.

Sensor 22 can consist of, but is not limited to, a thermocouple, a thermistor or phosphor coated optical fibers. The sensor 22 can be in an interior of the distal portion 20 or on the surface of the distal portion 20 and can further be a single sensor 22 or several sensors. It can also be a band or patch instead of a sensor 22 which senses only discrete points.

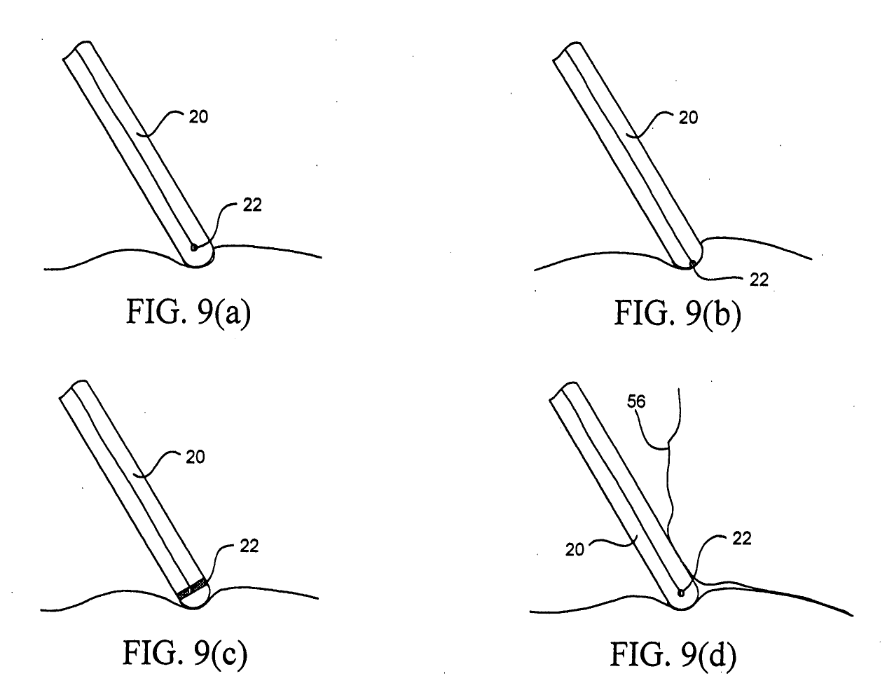

Sensor 22 provides a signal whose magnitude is representative of the thermal energy content of the surfaces and mediums in physical contact with the surface of the sensor 22. Thus, if several surfaces or mediums are in physical contact with sensor 22, the magnitude of the signal provided by sensor 22 will be representative of a composite of the thermal energy contents of those surfaces and/or mediums. Further, the effective surface of sensor 22 can be increased by wholly enclosing sensor 22 in a medium which easily conducts thermal energy. In this embodiment, thermal energy will be conducted from the surface of the thermally conductive medium to the sensor 22. The magnitude of the signal will represent a composite of the thermal energy contents of any surfaces and mediums in physical contact with the surface of the thermally conductive medium. For instance, FIG. 9(a) illustrates an embodiment where sensor 22 is located within distal portion 20. Further, distal portion 20 is in physical contact with a portion of the selected site 32 and fluid medium 30 adjacent to the selected site 32. Because of this contact, the thermal energy from selected site 32 and adjacent fluid medium 30 conducts through the thermally conductive sections of distal portion 20 to sensor 22. The magnitude of the resulting signal provided by sensor 22 represents the composite thermal energy content of selected site 32 and at least a portion of adjacent fluid medium 30.

By strategically positioning and configuring sensor 22, it is possible to design the distal portion 20 such that signal represents the thermal energy content of specific surfaces or mediums. For instance, FIG. 9(b) illustrates an embodiment where the sensor 22 is positioned such that the thermal energy conducted to the sensor is from substantially only selected site 32. Thus, sensor 22 provides signal which is representative of the thermal energy content of substantially only selected site 32. Further, FIG. 9(c) shows an embodiment where sensor 22 is configured as a band and positioned such that the thermal energy conducted to sensor is from substantially only fluid medium 30 adjacent to selected site 32. Thus, sensor 22 provides a signal which represents substantially only the thermal energy content of the adjacent fluid medium. FIG. 9(d) illustrates another embodiment where fluid medium adjacent to selected site 32 contains other tissue, organs or bodies 56. In this embodiment, the signal provided by sensor 22 represents a composite of the thermal energy contents of selected site 32 and adjacent fluid medium 30 as well as the other tissues, organs or bodies 56.

Sensor 22 provides the composite signal of thermal energy and temperature whether fluid medium 30 is flowing or non-flowing. When the surgeon chooses to deliver energy to selected area 34 by moving distal portion 20 from selected site 32 to another, it is possible to bring distal portion 20 into physical contact with a selected site 32 which has previously been elevated to the desired temperature for the desired period of time. This second application of energy may quickly elevate the temperature enough to cause cell necrosis or may cause the temperature at selected site 32 to remain elevated for longer than the desired period causing the collagen fibers to contract more than desired.

Positioning sensor 22 to provide a signal which represents a composite of the thermal energy contents of selected site 32 as well as adjacent fluid medium 30 reduces cell necrosis or over contraction caused by a second application of energy. As energy delivery device 14 delivers energy to selected site 32 it also delivers energy to fluid medium 30 which is in physical contact with energy delivery device 14 adjacent to selected site 32. This delivery of energy to fluid medium 30 causes the thermal energy content of fluid medium 30 to increase. The thermal energy content of fluid medium 30 adjacent to selected site 32 also rises due to conduction of thermal energy from selected site 32 to fluid medium 30. Furthermore, due to convection resulting from the movement of distal portion 20, thermal energy disperses through fluid medium 30 at a quicker rate than through collagen containing tissue 28.

As a result of the energy transfers described above, the corresponding elevations in temperature will be more dispersive in fluid medium 30 than in selected site 32. Thus, the signal produced by sensor 22 is different when distal portion 20 is placed adjacent to a previously heated selected site 32 than when distal portion 20 is placed in a selected site 32 away from any previously heated selected sites 32. Although the selected sites 32 in the former and latter cases will have similar thermal energies, in the former case, the dispersive energy in fluid medium 30 causes the fluid medium 30 to have a higher thermal energy content than in the latter case. Since sensor 22 provides a signal whose magnitude represents a composite of thermal energies of fluid medium 30 adjacent to selected site 32, in the former case sensor 22 provides a signal to feedback control system 40 indicating an elevated thermal energy content and reduces the amount of energy delivered to selected site 32. This reduced energy delivery decreases cell necrosis or overcontraction near selected site 32. The same is true in those instances when distal portion 20 is again passed over a previously heated selected site 32.

It will also be appreciated that when sensor 22 is positioned where it provides a signal representing a composite including adjacent fluid medium 30 when the surgeon chooses to paint selected area 34 rather than moving from one selected site 32 to another. The surgeon will want to keep the temperature of an entire selected area 34 within a specific range during the painting process. As distal portion 20 is painted across selected area 34 it leaves a path which has been heated and may intersect that path several times during the process of keeping the temperature within the desired range. As selected area 34 is covered and distal portion 20 intersects the heated path it is desirable to deliver more energy to areas which are not in the path and consequently have not been previously heated. It is also desirable to deliver less energy to areas which are part of the path and have previously been heated. By delivering energy this way, the thermal energy content of the selected area 34 will approach a uniform thermal energy across the selected area.

As described above, thermal energy can be more dispersive in fluid medium 30. As a result, when distal portion 20 is moved toward a previously heated path sensor 22 provides a different signal than it would if it were not traveling toward a previously heated path. Fluid medium 30 will have a higher thermal energy content in the former case than in the latter case. Since sensor 22 provides a signal whose magnitude is related to a composite of the thermal energies of fluid medium 30 adjacent to selected site 32 and the selected tissue site 32 in the former case sensor 22 provides a signal to feedback control system 40 indicating an elevated thermal energy content and reduces the amount of energy delivered to selected site 32. As a result of the elevated thermal energy content, feedback control system 40 reduces the amount of energy delivered in the former case. The result allows the temperature across the selected area 34 to approach uniformity. Uniformity of temperature is desirable as it reduces cell necrosis or overcontractions near path intersections.

Positioning sensor 22 such that it provides a signal which represents a composite of thermal energies including adjacent fluid medium 30 can also reduce stray contractions. Stray contractions are undesired contractions of collagen fibers outside selected area 34. As described above, while energy is delivered to selected area 34, the thermal energy content of fluid medium 30 also increases. During an extended treatment it is possible for the thermal energy content of fluid medium 30 to rise considerably. If the thermal energy content of fluid medium 30 remains elevated for an extended period of time it is possible for the conduction of thermal energy from fluid medium 30 to collagen containing tissue 28 to elevate the temperature of collagen containing tissue 28 sufficiently to cause undesired contractions of collagen fibers and may occur outside selected area 34. These stray contractions are even more of a problem when the fluid medium 30 is flowing since the flow will carry the heated fluid medium 30 away from the selected area.

These stray contractions are reduced by positioning sensor 22 to provide a composite signal which includes at least a portion of fluid medium adjacent to the selected site 32. For instance, when the thermal energy content of fluid medium 30 is raised, the signal will be different than when it is not and the energy delivery is adjusted accordingly. Since sensor 22 provides a signal whose magnitude is related to a composite which includes the fluid medium 30 adjacent to the selected site 32, in the former case sensor provides a signal to feedback control system 40 indicating an elevated thermal energy content and reduces the amount of energy delivered to selected site 32. The reduced energy delivery reduces the amount of energy delivered to fluid medium 30 and consequently reduce stray contractions.

Apparatus 10, comprising handpiece 12 and energy delivery device 14, is adapted to be introduced through an operating cannula 58 for percutaneous applications. It will be appreciated that apparatus 10 may be used in non-percutaneous applications and that an operating cannula 58 is not necessary in the broad application of the invention.

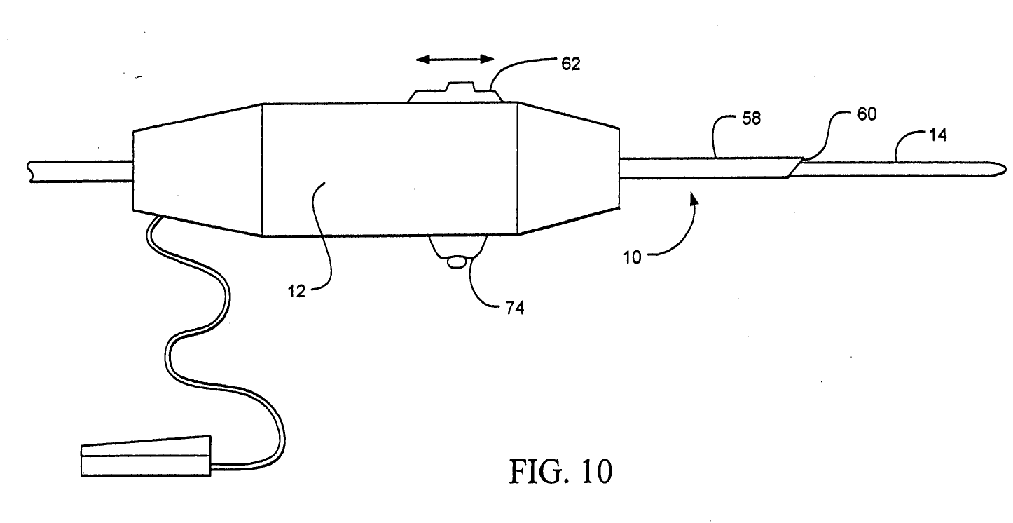

As illustrated in FIG. 10, apparatus 10 can also include, as an integral member, an operating cannula 58 which can be in the form of a hypodermic trocar with dimensions of about 3 to 6 mm outside diameter, with tubular geometries such as those of standard commercially available operating cannulas. Operating cannula 58 can be wade of a variety of biocompatible materials including but not limited to stainless steel, and the like.

Operating cannula 58 has a cannula proximal end that attaches to handpiece 12 and a cannula distal end 60 which can have a sharp or piercing end for penetrating body structures in order to introduce energy delivery device 14 to a selected site 32. Energy delivery device 14 is positioned within an interior lumen of operating cannula 58 and is extendable beyond cannula distal end 60 in order to reach selected site 32. Energy delivery device 14 can be advanced and retracted in and out of operating cannula 58 by activating a deployment button 62 which is located on the exterior of handle 12. Deployment button 62 is preferably activated by the operator merely by sliding it, which causes energy delivery device 14 to advance in a direction away from cannula distal end 60. Deployment button 62 can be pulled back, causing a retraction of energy delivery device 14 towards cannula distal end 60. In many instances, energy delivery device 14 is retracted to be positioned entirely within operating cannula 14. Energy delivery device 14 can also be deployed with fluid hydraulics, pneumatics, servo motors, linear actuators, and the like.

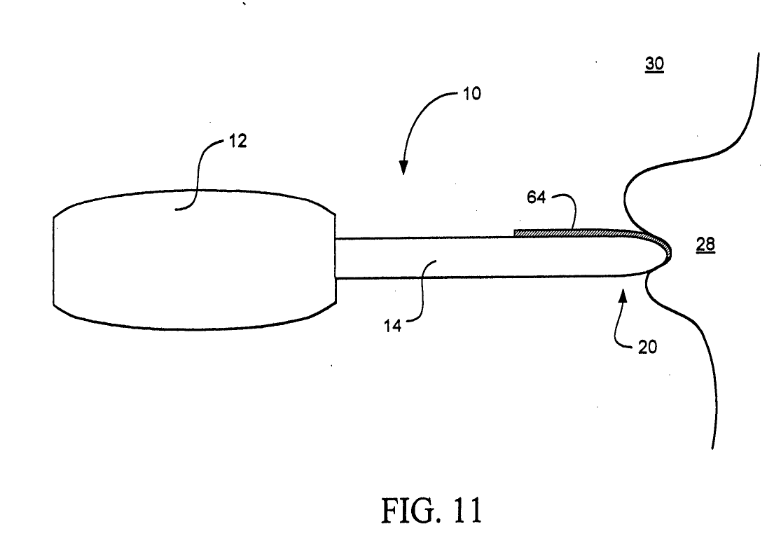

In FIG. 11, distal portion 20 of energy delivery device 14 includes an insulating layer 64 which is substantially impenetrable to the energy delivered to collagen containing tissue 28. Specifically, in the case of an RF energy source 24, electrical insulation can be used. Insulation 64 can be formed on energy delivery device 14 such that a minimum of energy is delivered to tissue, organs or other bodies which the surgeon does not wish to treat. For example, when energy delivery device 14 is introduced into a tight area, and only one surface of the tight area is to be treated, then it is desirable to avoid delivering energy outside of that surface. The inclusion of insulating layer 64 accomplishes this result. Suitable insulation materials include but are not limited to polyamide, epoxy varnish, PVC and the like.

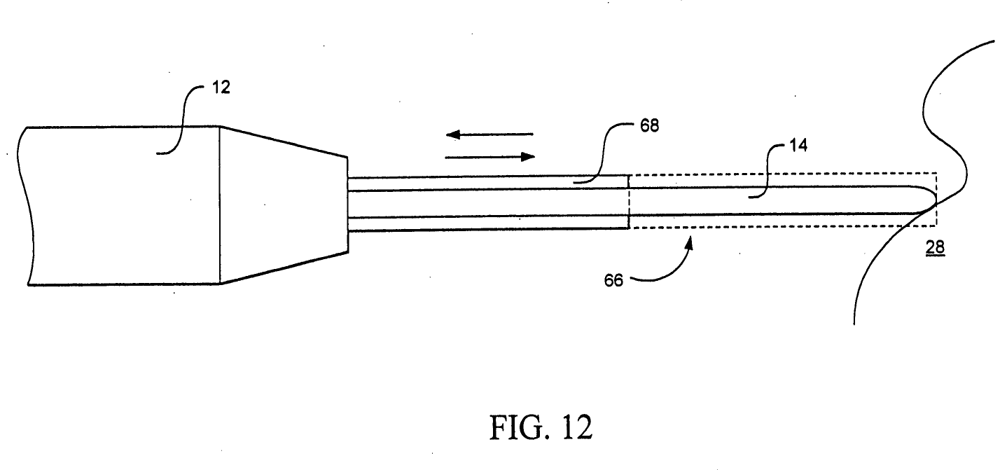

The area of energy delivery device 14 that serves as a conductive surface 66 can be adjusted by the inclusion of an insulating sleeve 68 (FIG. 12) that is positioned around energy delivery device 14. Sleeve 68 may be advanced and retracted along the surface of energy delivery device 14 in order to increase or decrease the surface area of conductive surface 44 that is directed to collagen containing tissue 28. Sleeve 68 can be made of a variety of materials including but not limited to nylon, polyamides, other thermoplastics and the like. The amount of available conductive surface 44 available to deliver thermal energy can be achieved with devices other than sleeve 68, including but not limited to printed circuitry with multiple circuits that can be individually activated, and the like.

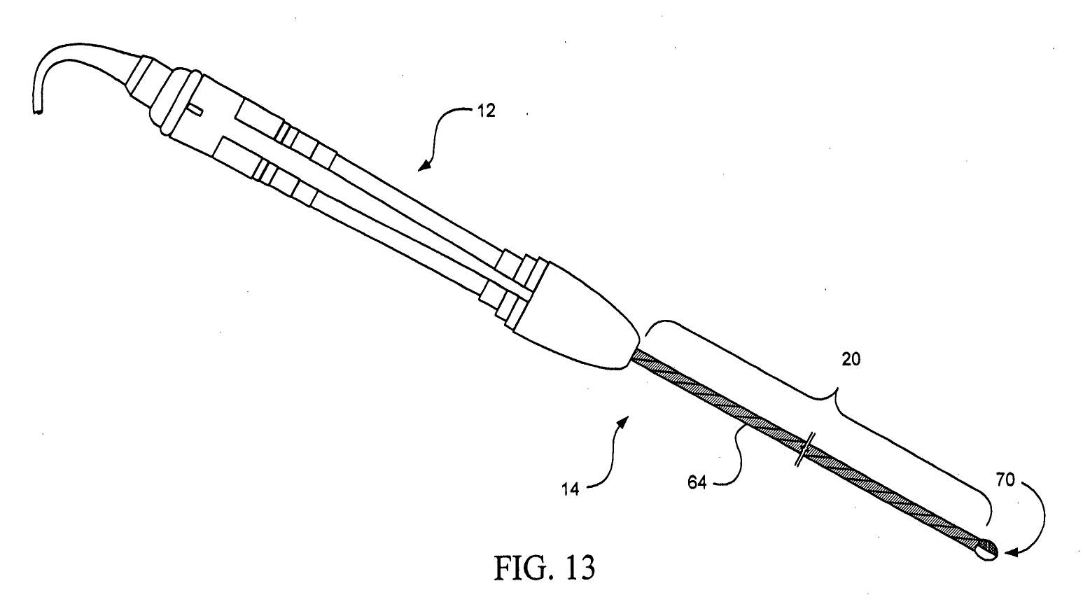

As illustrated in FIG. 13, distal portion 20 of energy delivery device 14 includes a thermally insulating layer 70 which is substantially impenetrable to thermal energy. Thus, thermal insulating layer 70 can be used to limit the amount of selected site 32 that contributes to the temperature detected by sensor 22. For example, by insulating only distal end 18 of distal portion 20 substantially only thermal energy from fluid medium 30 adjacent to selected site 32 is conducted to sensor 22. Thus, the magnitude of the signal produced by the sensor 22 represents substantially only the thermal energy concern of fluid medium 30 adjacent to selected site 32. Thermal energy insulating layer 70 can also be used in conjunction with a delivered energy insulating layer 64 to cover identical areas or different areas. Thermal insulating layer 70 can be constructed of the same material as the delivered energy insulating layer 64.

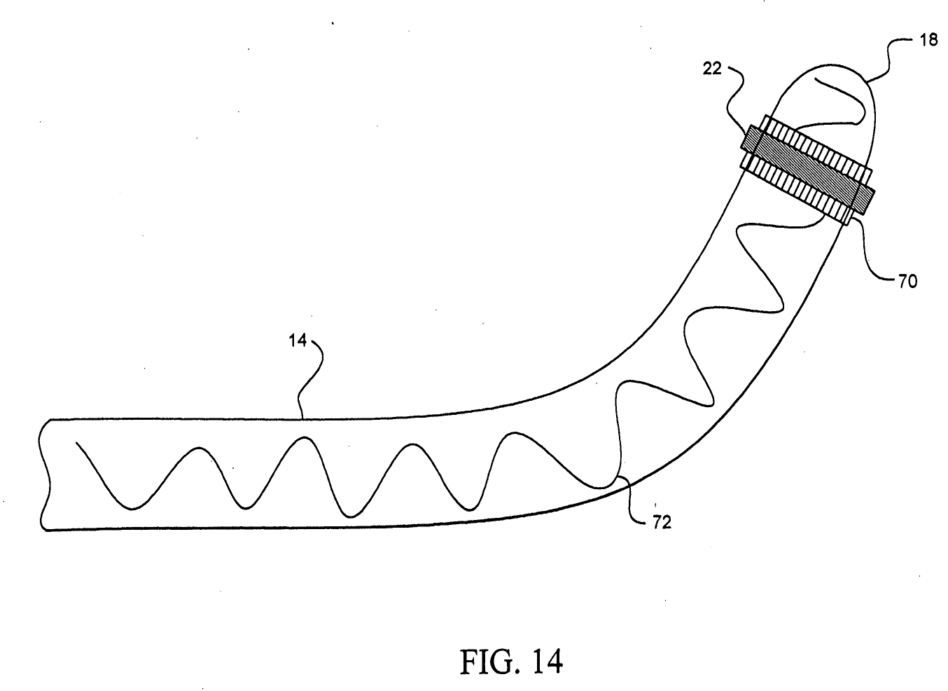

For many applications, it is necessary to have distal portion 20 become deflected. In FIG. 14, a resistive heating element 72 can be positioned in an interior lumen of energy delivery device 14 which is at least partially made of memory metal. Resistive heating element 72 can be made of a suitable metal that transfers heat to energy delivery device 14, causing distal portion 20 to become deflected when the temperature of energy delivery device 14 reaches a level that the memory metal is caused to deflect, as is well known in the art. Not all of energy delivery device 14 need be made of the memory metal. It is possible that only distal portion 20 be made of the memory metal in order to effect the desired deflection. When deflection is caused by heating memory metal, it is desirable to insulate sensor 22 from the effects of the resistive heating element 22. One method of doing this is demonstrated in FIG. 12 where thermal insulating layer 70 is located between the distal portion 20 and sensor 22 where sensor 22 is a band.

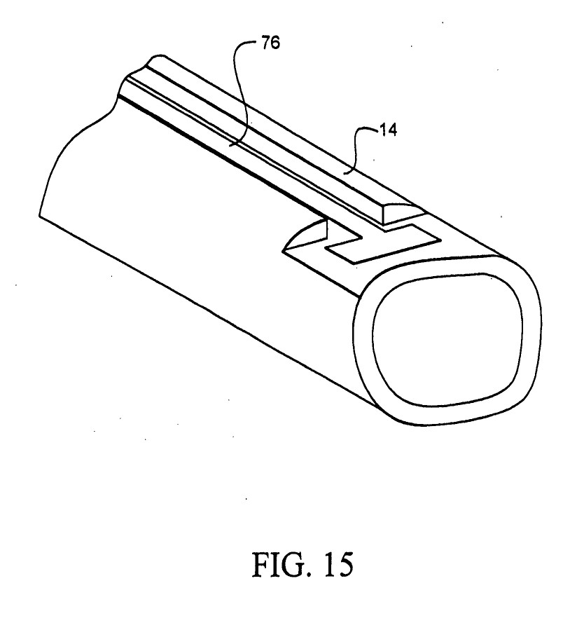

Deflection can also be accomplished mechanically. A steering wire, or other mechanical structure, is attached to either the exterior or interior of energy delivery device 14. A deflection button 74, located on handle 12 (FIG. 10), is activated by the physician, causing a steering wire 76 (FIG. 15) to tighten, and impart an retraction of energy delivery device 14, resulting in a deflection of distal portion 20. It will be appreciated that other mechanical mechanisms can be used in place of steering wire 76. The deflection may be desirable for selected sites 32 that have difficult access, and it is necessary to move about a non-planar collagen containing tissue 28. By deflecting distal portion 20, the opportunity to provide more even thermal energy to selected site 32 is achieved, and the possibility of ablating or dissociation of collagen material is greatly reduced.

As shown in FIG. 15, steering wire 76 attaches to a flat formed on the exterior of energy delivery device. Wire EDM technology can be used to form the flat on energy delivery device 14. A “T” bar configuration is illustrated in FIG. 15. Chemical etching may be used to create the T bar, Steering wire 76 need not be an actual wire. It can also be a high tensile strength cord such as Kevlar. Steering wire 76 can be made of stainless steel flat wire, sheet material, and the like.

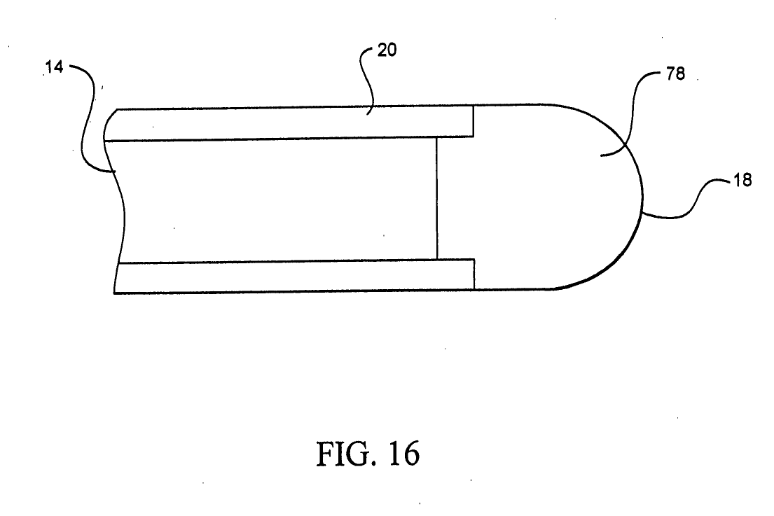

As shown in FIG. 16 energy delivery device 14 can be tubular in nature with a central lumen. Distal portion 20 can include a conductive plug 78 that is sealed to distal portion 24 by welding, e-beam, laser and the like.

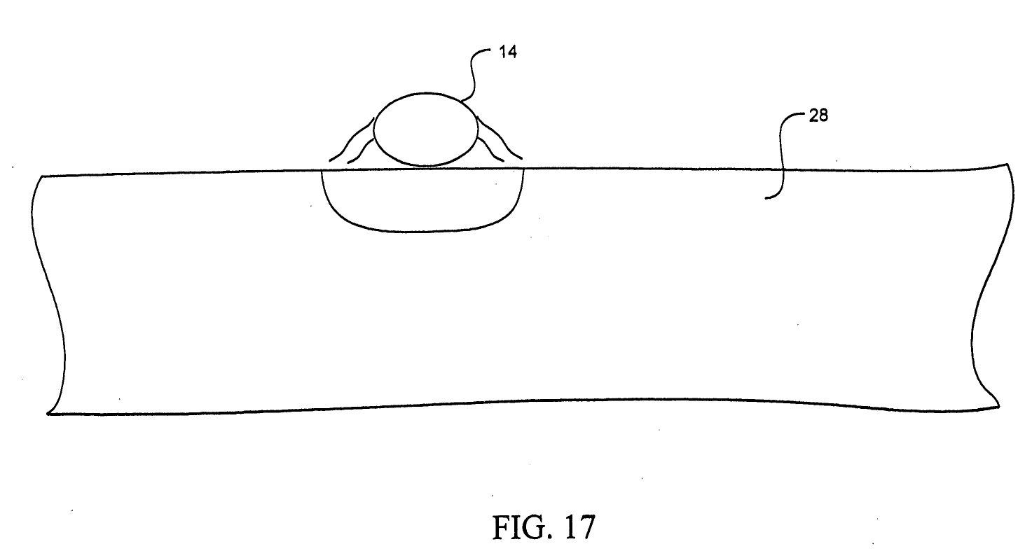

Energy delivery device 14 can have a variety of different geometric configurations which can vary based on the type and shape of collagen containing tissue 28 to be heated. In FIG. 17, energy delivery device 14 has an oval cross section. The oval cross section provides a greater conductive surface 66 area that is in contact with collagen containing tissue 28. A larger zone of heating to collagen containing tissue 28 is provided. The thermal gradient within collagen containing tissue 28 is more even and the possible dissociation or breakdown of the collagen fibers is reduced.

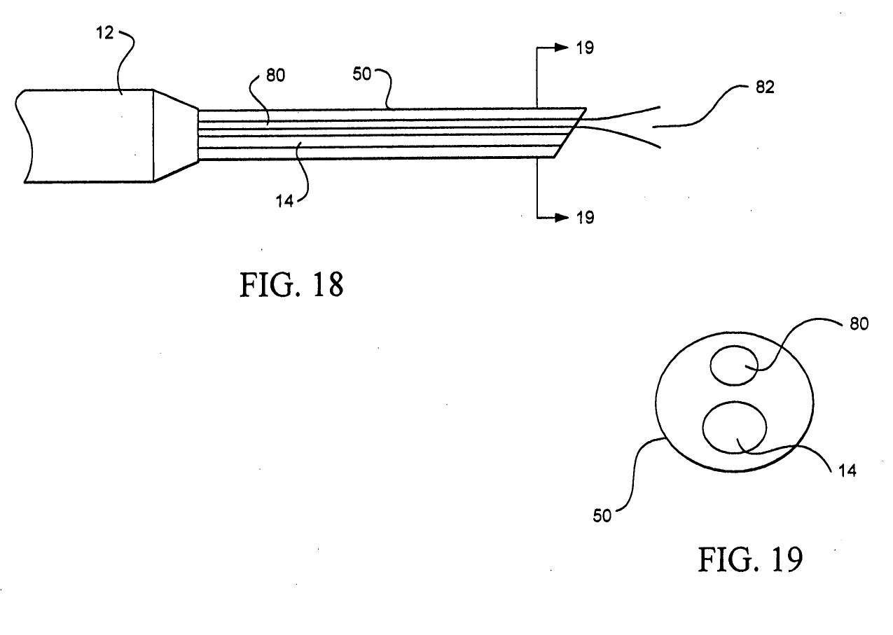

As illustrated in FIGS. 18 and 19, operating cannula 58 may include a viewing scope 80 which may be positioned adjacent to energy delivery device 14. Viewing scope 80 provides a field of view 82, permitting the surgeon to view while delivering energy to selected site 32 and contracting collagen containing tissue 28. Viewing scope 80 can include a bundle of light transmitting fibers and optical viewing elements. Alternatively, the surgeon can view the procedure under arthroscopic visualization.

The present invention also provides a method of contracting collagen containing tissue 28. The collagen containing tissue 28 is contracted to a desired shrinkage level while minimizing cell necrosis as well as damage to surrounding organs and other bodies. It can be used in the joints such as the shoulder, spine, cosmetic applications, and the like. It will be appreciated to those skilled in the art that the present invention has a variety of different applications, not merely those specifically mentioned in this specification. Some specific applications include joint capsules, specifically the gleno-humoral joint capsule of the shoulder, herniated discs, the meniscus of the knee, in the bowel, for hiatal hernias, abdominal hernias, bladder suspensions, tissue welding, DRS, and the like.

The surgeon determines which collagen containing tissues 28 require contraction and how much shrinkage should occur. The surgeon then selects an area of the collagen containing tissue 28 for shrinkage. The surgeon can find the selected area 34 by using arthroscopic viewing or using the apparatus 10 include a viewing scope 80. Once the surgeon places the energy delivery device 14 next to the selected site 32, the surgeon soon begins delivery of energy.

While embodiments and applications of this invention have been shown and described, it will be apparent to those skilled in the art that many more modifications than mentioned above are possible without departing from the invention concepts herein. The invention, therefore, is not to be restricted except in the spirit of the appended claims.

1. A cosmetic treatment method comprising:
providing a treatment apparatus comprising a microprocessor, an energy source and energy delivery device coupled to the energy source, said energy delivery device comprising an electrode and at least one sensor associated with the electrode;
providing a fluid medium to contact at least a portion of the electrode;
placing the electrode on a surface of a tissue site without penetrating the surface;
determining with the sensor at least one composite temperature of the tissue site and the fluid medium in contact with the electrode;
sending a signal indicative of the composite temperature from the sensor to the microprocessor; and
delivering energy from the electrode to the tissue site.

2. The cosmetic treatment method of claim 1 further comprising:
contracting collagen fibers in the tissue site.

3. The cosmetic treatment method of claim 2 further comprising:
contracting at least a portion of the tissue site.

4. The cosmetic treatment method of claim 1 wherein said step of delivering energy comprises delivering energy from the electrode to the tissue site while minimizing ablation of the tissue site.

5. The cosmetic treatment method of claim 1 further comprising:
measuring current supplied to the energy delivery device.

6. The cosmetic treatment method of claim 5 further comprising:
displaying the current supplied to the energy delivery device.

7. A cosmetic system for contracting collagen fibers in a tissue site, said apparatus comprising:
a fluid medium;
an energy source; and
an energy delivery device coupled to the energy source, said energy delivery device comprising
a distal end having a conductive material in contact with the fluid medium and a non-conductive material in contact with at least a portion of the tissue site;
an internal lumen extending from the conductive material toward a proximal end; and
a sensor associated with the distal end and in contact with the fluid medium,
wherein the energy delivery device is configured to be positioned adjacent to the tissue site such that the sensor determines at least one composite temperature of the tissue site and the fluid medium.

8. The cosmetic system of claim 7 wherein the energy source is an RF energy source.

9. The cosmetic system of claim 7 further comprising:
a microprocessor wherein the sensor is configured to provide a signal indicative of the composite temperature to the microprocessor.

10. The cosmetic system of claim 7 wherein the sensor is positioned on a surface of the distal end.

11. The cosmetic system of claim 7 wherein the sensor is positioned on the conductive material.

12. The cosmetic system of claim 11 wherein the sensor is positioned within the internal lumen.

13. The cosmetic system of claim 7 wherein the energy source and the energy delivery device are coupled by a cable.

14. The cosmetic system of claim 7 wherein the fluid medium is a liquid.

15. The cosmetic system of claim 14 wherein the fluid medium is in contact with the sensor.

16. The cosmetic system of claim 7 further comprising a handle coupled to the proximal end of the energy delivery device.

17. The cosmetic system of claim 7 wherein at least a portion of the conductive material is positioned between the internal lumen and the non-conductive material.
